(19) United States
(12) Patent Application Publication
(10) Pub. No.: US 2005/0014250 A1
(43) Pub. Date: Jan. 20, 2005

(54) Bioartificial organ for hosting animal and/or human cells

(76) Inventor: Daniele Galavotti, Mirandola (IT)

Correspondence Address: Modiano & Associati, Via Meravigli, 16, 20123 Milano, IT

(21) Appl. No.: 10/362,601

(22) PCT Filed: Aug. 28, 2001
(86) PCT No.: PCT/EP01/09906

(30) Foreign Application Priority Data
Sep. 1, 2000 (IT) MO2000A000182

(51) Int. Cl. C12M 3/04

(52) U.S. Cl. 435/297.2; 435/297.4; 435/299.1

(57) ABSTRACT

A bioartificial organ for hosting animal and/or human cells comprising a container body having an internal cavity which can accommodate a cell support and culture structure; the body is provided with at least one inlet port for plasma or ultrafiltrate to be treated drawn from a patient, the inlet port being arranged upstream of the support and culture structure, and with an outlet port for the treated plasma or ultrafiltrate, which is arranged downstream of the support and culture structure; between the structure and the internal cavity two end chambers are provided, respectively a first chamber for collecting plasma or ultrafiltrate to be treated and a second chamber for collecting treated plasma or ultrafiltrate; the chambers are connected to the outside through the ports, and a shaft-like element for coupling the support and culture structure is further provided inside the cavity of the container body and is arranged between the ends of the container body.

### BACKGROUND OF THE INVENTION

The present invention relates to a bioartificial organ for hosting animal and/or human cells.

Bioartificial organs and devices used for clinical applications in humans have long been known.

In particular, the development and clinical use of models of bioreactors capable of hosting, with several culture systems, mammalian cells capable of reproducing specific functions of an organ to be assisted have become highly important in this context.

Even more specifically, particular attention has been devoted to the study of bioartificial liver models that allow to host and cultivate animal hepatocytes with a protein synthesis and a metabolism that are effective and constant over time.

The intended goal of these studies and of the resulting structural prototypes is the common intent to devise a three-dimensional support being capable of hosting said hepatocytes arranged according to a preset geometry capable of allowing perfusion by the plasma of the patient so that the cultured cells can exchange with said plasma solutes and gases dissolved therein, while preventing the physical passage of the cells, or even of fragments thereof, into said plasma, in order to avoid immunization phenomena.

The first devices used in the clinical field and intended specifically for liver assistance have used the principle of diffusion and exchange of solutes with different molecular weights through a membrane, drawing this concept directly from dialysis and dialyzers.

These devices have evolved from an initial version with a flat membrane to the more recent ones which use hollow-fiber culture structures capable of accommodating the cell cultures externally or internally.

The adoption of capillary fibers has allowed to optimize the solute exchange and diffusion processes, significantly improving the fluid-dynamics characteristics of bioreactors built with this technique; moreover, the combination of different geometrical structures or of structures having a plurality of microporous capillary bundles has allowed to provide the necessary supply of oxygen, which is essential for cell metabolism.

However, these prototypes of the so-called “bioartificial liver” have always suffered from drawbacks which are described hereinafter.

A first drawback is the need to achieve effective exchange between the plasma of the patient and the cells contained in the culture device, so that this exchange occurs constantly over the entire exchange surface. In conventional devices, this exchange is limited by the low pressure at which the plasma or ultrafiltrate, arriving from the patient, is introduced in the device; such low pressure must be maintained in order to avoid submitting the hepatocytes to pressure shocks caused by high supply pressures of the plasma or ultrafiltrate, compromising their vitality and ultimately their functionality.

A second drawback is the possibility to have an adequate volume of cells, so as to sufficiently replace the hepatic function in the human body; in conventional devices, such volume is very small in relation to their overall dimensions and to the need to assist the hepatic function: in conventional devices, in fact, the useful volume for culture is approximately ⅓ of the total volume of the device, whereas 200 to 300 grams of hepatocytes are necessary in order to support the hepatic functionality of an adult.

A third drawback is constituted by the fact that the device and the cells cultivated therein must allow perfusion uniformly along the entire extension of the culture, avoiding, as occurs in conventional devices, a concentration of exchange in the inlet region to the detriment of the terminal outlet regions and ultimately a partial utilization of the device. This occurs because conventional devices contain culture supports which are organized randomly in terms of geometry, making it troublesome, as mentioned, to maintain constant perfusion.

A fourth drawback is that the method of preparing the bioartificial organ must be safe, rapid and repeatable as regards its effectiveness, in order to minimize the time required for clinical use, which is aimed mainly at acute conditions in which the time factor becomes vitally important.

A fifth drawback resides in that the device must allow a perfusion that prevents any possible passage of cells or fragments thereof into the circulation of the patient.

### SUMMARY OF THE INVENTION

The aim of the present invention is to eliminate the above-noted drawbacks of the prior art by providing a bioartificial organ for hosting animal and/or human cells that solves all the above listed technical drawbacks.

This aim and these and other objects which will become better apparent hereinafter are achieved by a bioartificial organ for hosting animal and/or human cells, comprising a container body having an internal cavity which can accommodate a cell support and culture structure, said body being provided with at least one inlet port for plasma or ultrafiltrate to be treated drawn from a patient, said inlet port being arranged upstream of said support and culture structure, and with an outlet port for said treated plasma or ultrafiltrate which is arranged downstream of the support and culture structure, characterized in that two end chambers are formed between said structure and said internal cavity, respectively a first chamber for collecting plasma or ultrafiltrate to be treated and a second chamber for collecting treated plasma or ultrafiltrate, said chambers being connected to the outside through said ports, a shaft-like element for coupling said support and culture structure being further provided inside said cavity of the container body and being arranged between the ends of said container body.

### BRIEF DESCRIPTION OF THE DRAWINGS

Further characteristics and advantages of the present invention will become better apparent from the description of a preferred embodiment of a bioartificial organ for hosting animal and/or human cells, illustrated only by way of non-limitative example in the accompanying drawings, wherein:

FIG. 1 is a schematic sectional view, taken along a longitudinal plane, of the bioartificial organ according to the invention;

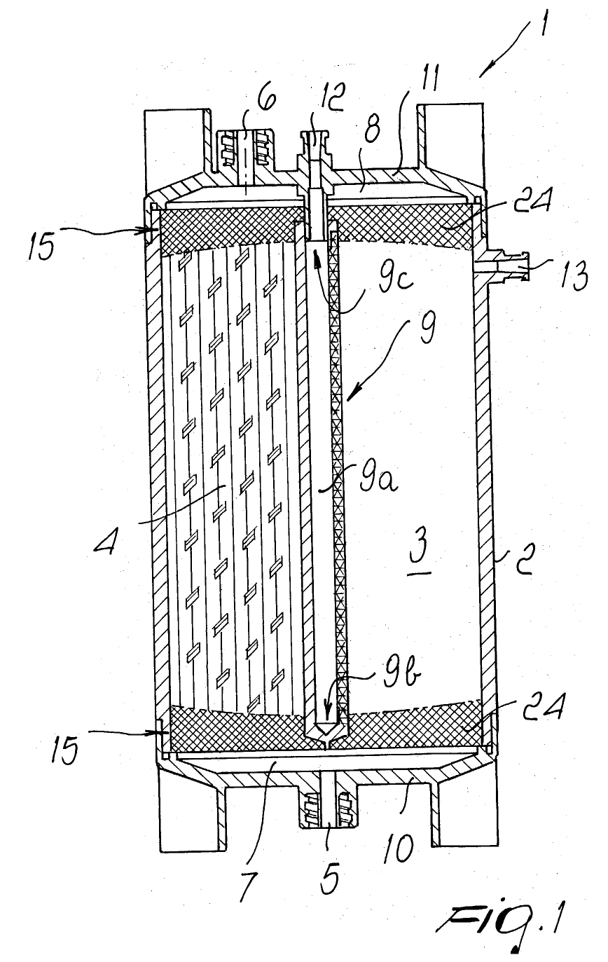

FIG. 2 is a highly enlarged-scale detail view of a portion of the cell support and culture structure;

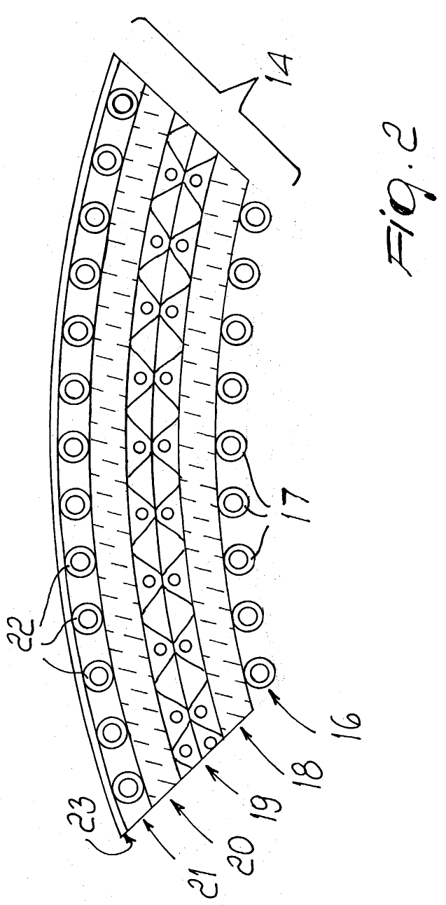

FIG. 3 is an end view of the cell support and culture structure in the region for coupling to a centered shaft-like element with which the bioartificial organ according to the invention is provided internally;

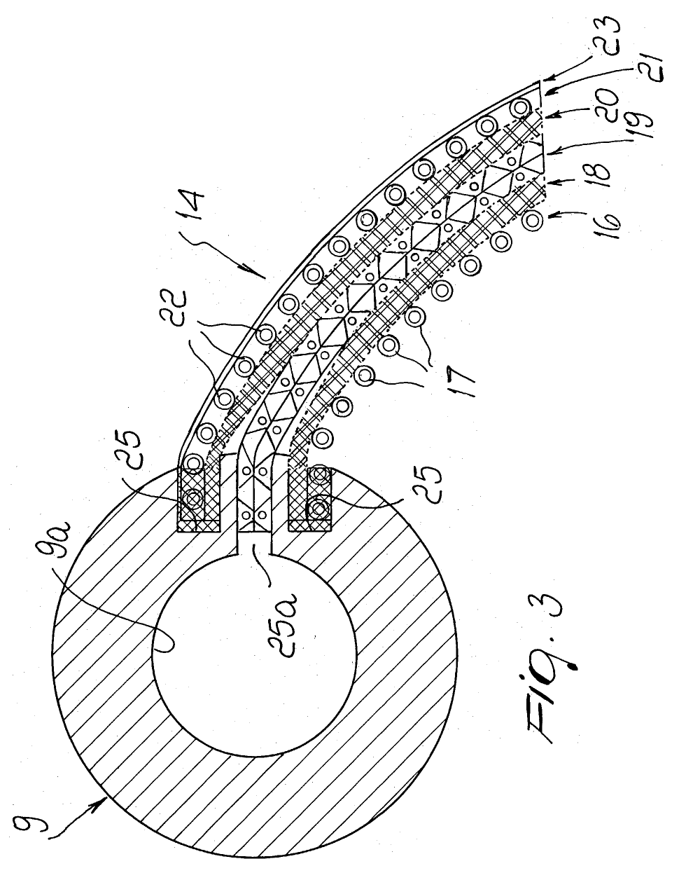

FIG. 4 is a perspective view, with parts in phantom lines, of a portion of said cell support and culture structure.

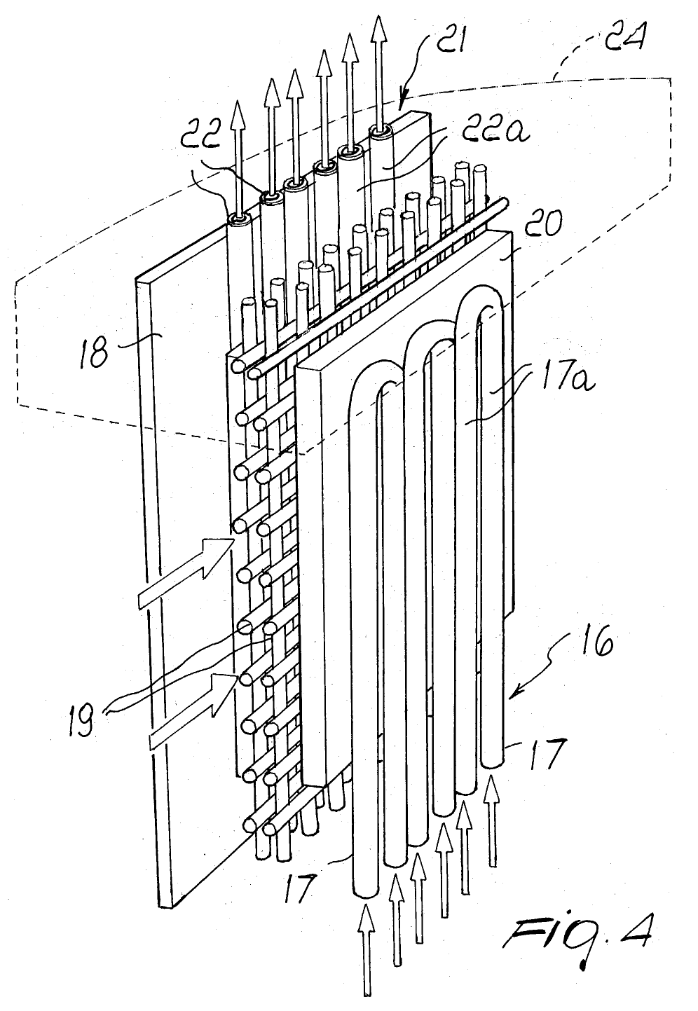

### DESCRIPTION OF THE PREFERRED EMBODIMENTS

With reference to the figures, the reference numeral 1 designates a bioartificial organ for hosting animal and/or human cells, which comprises a substantially cylindrical container body 2, having an internal cavity 3 which can accommodate a cell support and culture structure 4.

The container body 2 is provided with at least one inlet port 5 for plasma or ultrafiltrate drawn from a patient, which is arranged upstream of the structure 4, and with an outlet port 6 for the treated plasma or ultrafiltrate, arranged downstream of the structure 4.

Between the outlet port and the internal cavity 3 two end chambers are provided, respectively a first chamber 7 for collecting plasma or ultrafiltrate to be treated and a second chamber 8 for collecting treated plasma or ultrafiltrate, such chambers being connected to the outside through the ports 5 and 6.

Moreover, inside the cavity 3 a shaft-like element 9 for the coupling of the structure 4 is also provided which is arranged between the ends of the container body 2; such ends of the container body are constituted by respective hermetic closure caps 10 and 11, in each of which the ports 5 and 6 are formed.

The shaft-like element 9, which is fitted coaxially and centered in the cavity 3, is crossed axially and along its entire length by a cavity 9a and has a blind end 9b and an open end 9c connected to the outside through its own opening 12 which passes through the second chamber 8 and the corresponding cap 11.

The cavity 3 also is directly connected to the outside through a second opening 13, which passes through the side wall of the container body 2.

The cell support and culture structure 4 is constituted by a permeable coil 14 composed of a spiral winding, around the shaft-like element 9, of a multilayer fabric packed in a sandwich-like configuration; the leading edge of said winding is fixed to the shaft-like element 9 so that at least one layer, specified hereinafter, is connected to the axial cavity 9a thereof, while the trailing edge remains in contact with the internal wall of said axial cavity 3 of the container body 2; the coil 14 is embedded in two end containment means 15.

The fabric composing the coil 14 is composed in a modular fashion of at least six stacked sheet-like layers. A first innermost layer is constituted by a first flat ordered arrangement 16 of capillary fibers 17 arranged parallel to the longitudinal axis of said container body 2 and folded in a U-like shape, the free ends of which pass through the respective containment means 15 and lead into the second collection chamber 8. A second layer is constituted by a permeable and filtering sheet-like means 18 which is adapted to support the cells. A third layer is constituted by a sheet-like grid 19 which is adapted to distribute the cells to be seed. A fourth layer is constituted by a second permeable and filtering sheet-like means 20 which is again adapted to support the cells. A fifth layer is constituted by a second flat ordered arrangement 21 of capillary fibers 22, which are parallel and opposite to the fibers 17 of the first ordered arrangement 16, are also folded in a U-like shape and have free ends that pass through the respective containment means 15 and lead into the first collection chamber 7. A sixth outermost layer is constituted by a separator sheet 23 made of impermeable material.

The permeable and filtering sheet-like means 18 and the second one 20 are both constituted by sheets of polymeric fabric, preferably polyester, whose weft is woven with a random or ordered arrangement.

The end containment means 15 are constituted by two rings 24 made of a composite material based on polyurethane and formed snugly transversely inside the cavity 3: the rings form, together with said caps 10 and 11, the first collection chamber 7 and the second collection chamber 8.

The leading edge of the winding that constitutes the coil 14 is fixed to the shaft-like element 9 after interlocking and gluing in a slotted seat 25 formed along its entire length; the seat 25 has a central portion 25a which is connected, along its entire length, to the axial cavity 9a; the leading edge of the sheet-like distribution grid 19 that constitutes the third layer of the fabric is inserted in the portion 25a. In detail, each capillary fiber 17 and 22 has a constant diameter and is constituted by a tube-shaped segment of microporous material, preferably polyethersulfone, with pores having diameters between 0.10 and 0.50 microns, and, as mentioned, is folded in a U-like shape substantially at its centerline, so as to form a pair of straight and parallel branches 17a, 22a being directed in mutually opposite directions and so that the ports of the respective free ends match up and lead into a corresponding collection chamber for plasma or ultrafiltrate, the chamber 7 for the fibers 17 and the chamber 8 for the fibers 22.

The distribution of the fibers 17 and 22 also is constant along the entire extension of the coil 14.

The operation of the bioartificial organ according to the invention is as follows: the cells to be supported and cultured are introduced in the bioartificial organ 1 through the opening 12, which conveys them into the axial cavity 9a of the shaft-like element 9.

From there, through the central portion 25a of the seat 25, which is connected along its entire length with it and in which the leading edges of the layers composing the permeable coil 14 are interlocked and glued, said cells diffuse into the third layer 19 constituted by a sheet-like grid with interwoven fibers.

The coil 14 is wound in a spiral around the shaft-like element 9 and is fully contained in the axial cavity of the container body 2, firmly retained at its ends by the rings 24 in which said ends are embedded in order to keep said spiral wrapping firm.

Both rings 24 are crossed only by the ends of the capillary fibers 17 and 22, which converge into the respective chambers for collecting plasma or ultrafiltrate.

After cell insertion has been completed by following the helical path of the wrapping, which arranges them in a constant fashion inside the coil 14, said cells diffuse with a radial flow from the third layer 19 to the adjacent layers 18 and 20, both constituted by a sheet of woven polymeric fabric to which the cells adhere, producing the culture of said cells.

In this state, the bioartificial organ 1 can be used on a patient: plasma or the so-called ultrafiltrate is in fact drawn from said patient and introduced in said organ through the port 5 by means of a circuit operating at a low pressure provided by a conventional pumping means and is collected from there in the chamber 7, into which the openings of the ordered arrangement 16 of capillary fibers 17 composing the coil 14 converge.

The plasma or ultrafiltrate flows through the fibers 17 folded in a U-like arrangement and once it has saturated them it passes through their porous walls, moving with a radial flow towards the layer 18 on which the cells are supported.

By passing through such layer, the plasma or ultrafiltrate is purified by contact with the cells, maintaining a flow which is substantially perpendicular to the layers that compose the coil 14 until it reaches the second ordered arrangement 21 of capillary fibers 22, after passing through the next sheet 20 which acts, like the sheet 18, both as a support and as a filter for the cells, in order to prevent any of said cells, or even fragments thereof, from entering the blood circuit of the patient.

When the flow of plasma or ultrafiltrate has reached the second order arrangement 21 of capillary fibers 22, it is retained by the sheet 23 made of impermeable material and is forced to penetrate through the porous walls thereof into the openings of the fibers; it is propelled through such openings into the collection chamber 8 and from there finally returned to the patient through the port 6 in an already purified state.

The above described path is followed for all the turns that compose the coil 14, making the exchange between plasma or ultrafiltrate and the cells extremely uniform over the entire useful volume of the axial cavity 3 of the container body 2.

The second opening 13 formed in the lateral wall of the container body 2 allows, during seeding of the cells in the coil 14, the recirculation of the solution that contains them, after connection, by means of a tube provided with a pump (both not shown because of a conventional type), to the opening 12: once seeding has been completed, the opening 12 and the opening 13 are closed by means of convenient plugs.

In practice it has been found that the described invention achieves the intended aim and objects.

The invention thus conceived is susceptible of numerous modifications and variations, all of which are within the scope of the appended claims, so that it can optionally be used also in combination with conventional devices for oxygenation or adsorption of endogenous substances present in the plasma or ultrafiltrate.

All the details may further be replaced with other technically equivalent ones.

In practice, the materials used, as well as the shapes and the dimensions, may be any according to requirements without thereby abandoning the scope of the protection of the appended claims.

The disclosures in Italian Patent Application No. M02000A000182 from which this application claims priority are incorporated herein by reference.

1. A bioartificial organ for hosting animal and/or human cells, comprising a container body having an internal cavity which can accommodate a cell support and culture structure, said body being provided with at least one inlet port for plasma or ultrafiltrate to be treated drawn from a patient, said inlet port being arranged upstream of said support and culture structure, and with an outlet port for said treated plasma or ultrafiltrate which is arranged downstream of the support and culture structure, characterized in that two end chambers are formed between said structure and said internal cavity, respectively a first chamber for collecting plasma or ultrafiltrate to be treated and a second chamber for collecting treated plasma or ultrafiltrate, said chambers being connected to the outside through said ports, a shaft-like element for coupling said support and culture structure being further provided inside said cavity of the container body and being arranged between the ends of said container body.

2. The bioartificial organ according to claim 1, characterized in that the ends of said container body are constituted by respective hermetic closure caps, in each of which said inlet and outlet ports are respectively formed.

3. The bioartificial organ according to claim 1, characterized in that said shaft-like element is axially hollow and has a blind end and an open end which is connected to the outside through an opening that passes through said second chamber and the corresponding cap.

4. The bioartificial organ according to claim 1, characterized in that said shaft-like element is mounted so as to be centered coaxially in said cavity of the container body.

5. The bioartificial organ according to claim 1, characterized in that said cavity of the container body is directly connected to the outside through a second opening which passes through the side wall of said container body for direct short-circuiting connection of said cavity of said container body to said cavity of said shaft-like element during the seeding of the cells to be cultivated.

6. The bioartificial organ according to claim 1, characterized in that said cell supporting and culture structure is constituted by a permeable coil which is composed of a spiral winding of a multilayer fabric packed in a sandwich-like configuration, the leading edge of said winding being fixed to said shaft-like element on which said winding is wound with at least one layer connected to the axial cavity thereof, the trailing edge of said wrapping being in contact with the inner wall of said axial cavity of the container body, said coil being embedded in two end containment means.

7. The bioartificial organ according to claim 6, characterized in that said multilayer fabric is composed in a modular fashion of at least six superimposed sheet-like layers: a first innermost layer, constituted by a first flat ordered arrangement of capillary fibers arranged parallel to the longitudinal axis of said container body and folded in a U-like shape so that their free ends pass through the respective containment means and end in said second collection chamber; a second layer, constituted by a permeable and filtering sheet-like means for supporting the cells; a third layer, constituted by a sheet-like grid for distributing the cells to be seeded; a fourth layer, constituted by a second sheet-like permeable and filtering means for supporting the cells; a fifth layer, constituted by a second ordered arrangement of capillary fibers which are parallel and opposite to the fibers of said first layer, are folded in a U-like shape and have free ends which pass through the respective containment means and lead into said first collection chamber; a sixth outermost layer, constituted by a separator sheet made of impermeable material.

8. The bioartificial organ according to claim 6, characterized in that said permeable coil is wound through an arc of at least 270° with respect to said leading edge of the winding.

9. The bioartificial organ according to claim 7, characterized in that said first and second sheet-like means for cell support are constituted by sheets made of random-woven polymeric fabric.

10. The bioartificial organ according to claim 7, characterized in that said first and second sheet-like means for cell support are constituted by sheets made of orderly-woven polymeric fabric.

11. The bioartificial organ according to claim 10, characterized in that said polymeric fabric of said first and second sheet-like means is constituted by polyester.

12. The bioartificial organ according to claim 9, characterized in that the overall volume of said first and second sheet-like means for cell support is between 5 and 15% of the overall volume available for the mass of said cells.

13. The bioartificial organ according to claim 6, characterized in that said end containment means are constituted by two rings made of compound material based on polyurethane, which are arranged or formed transversely and snugly inside said cavity of the container body and form, together with said caps, said first and second collection chambers.

14. The bioartificial organ according to claim 6, characterized in that said leading edge of said winding is fixed to said shaft-like element after interlocking and gluing in a slotted seat formed along its entire length.

15. The bioartificial organ according to claim 14, characterized in that said seat has a central portion which is connected, along its entire length, to the axial cavity of said shaft-like element, the leading edge of said sheet-like distribution grid that forms said third layer being inserted in said central portion.

16. The bioartificial organ according to claim 7, characterized in that each capillary fiber is constituted by a tube-like segment of microporous material which is folded in a U-like shape substantially at the centerline so as to form two straight and parallel branches in which the openings of the matching free ends lead into a corresponding collection chamber for plasma or ultrafiltrate.

17. The bioartificial organ according to claim 16, characterized in that said microporous material has pores with a diameter between 0.10 and 0.50 microns.

18. The bioartificial organ according to claim 16, characterized in that said microporous material is constituted by polyethersulfone.

19. The bioartificial organ according to claim 7, characterized in that the distribution density and the diameter of said capillary fibers are constant over the entire extension of said coil that constitutes said cell support and culture structure.

20. A bioartificial organ for hosting animal and/or human cells, comprising a container body having an internal cavity which can accommodate a cell support and culture structure, said body being provided with at least one inlet port for plasma or ultrafiltrate to be treated drawn from a patient, said inlet port being arranged upstream of said support and culture structure, and with an outlet port for said treated plasma or ultrafiltrate which is arranged downstream of the support and culture structure, wherein two end chambers are formed between said structure and said internal cavity, respectively a first chamber for collecting plasma or ultrafiltrate to be treated and a second chamber for collecting treated plasma or ultrafiltrate, said chambers being connected to the outside through said ports, a shaft-like element for coupling said support and culture structure being further provided inside said cavity of the container body and being arranged between the ends of said container body.

21. The bioartificial organ according to claim 20, wherein the ends of said container body are constituted by respective hermetic closure caps, in each of which said inlet and outlet ports are respectively formed.

22. The bioartificial organ according to claim 20, wherein said shaft-like element is axially hollow and has a blind end and an open end which is connected to the outside through an opening that passes through said second chamber and the corresponding cap.

23. The bioartificial organ according to claim 20, wherein said shaft-like element is mounted so as to be centered coaxially in said cavity of the container body.

24. The bioartificial organ according to claim. 20, wherein said cavity of the container body is directly connected to the outside through a second opening which passes through the side wall of said container body for direct short circuiting connection of said cavity of said container body to said cavity of said shaft-like element during the seeding of the cells to be cultivated.

25. The bioartificial organ according to claim 20, wherein said cell supporting and culture structure is constituted by a permeable coil which is composed of a spiral winding of a multilayer fabric packed in a sandwich-like configuration, the leading edge of said winding being fixed to said shaft-like element on which said winding is wound with at least one layer connected to the axial cavity thereof, the trailing edge of said wrapping being in contact with the inner wall of said axial cavity of the container body, said coil being embedded in two end containment means.

26. The bioartificial organ according to claim 25, wherein said multilayer fabric is composed in a modular fashion of at least six superimposed sheet-like layers: a first innermost layer, constituted by a first flat ordered arrangement of capillary fibers arranged parallel to the longitudinal axis of said container body and folded in a U-like shape so that their free ends pass through the respective containment means and end in said second collection chamber; a second layer, constituted by a permeable and filtering sheet-like means for supporting the cells; a third layer, constituted by a sheet-like grid for distributing the cells to be seeded; a fourth layer, constituted by a second sheet-like permeable and filtering means for supporting the cells; a fifth layer, constituted by a second ordered arrangement of capillary fibers which are parallel and opposite to the fibers of said first layer, are folded in a U-like shape and have free ends which pass through the respective containment means and lead into said first collection chamber; a sixth outermost layer, constituted by a separator sheet made of impermeable material.

27. The bioartificial organ according to claim 25, wherein said permeable coil is wound through an arc of at least 270° with respect to said leading edge of the winding.

28. The bioartificial organ according to claim 26, wherein said first and second sheet-like means for cell support are constituted by sheets made of random-woven polymeric fabric.

29. The bioartificial organ according to claim 26, wherein said first and second sheet-like means for cell support are constituted by sheets made of orderly-woven polymeric fabric.

30. The bioartificial organ according to claim 29, wherein said polymeric fabric of said first and second sheet-like means is constituted by polyester.

31. The bioartificial organ according to claim 28, wherein the overall volume of said first and second sheet-like means for cell support is between 5 and 15% of the overall volume available for the mass of said cells.

32. The bioartificial organ according to claim 25, wherein said end containment means are constituted by two rings made of compound material based on polyurethane, which are arranged or formed transversely and snugly inside said cavity of the container body and form, together with said caps, said first and second collection chambers.

33. The bioartificial organ according to claim 25, wherein said leading edge of said winding is fixed to said shaft-like element after interlocking and gluing in a slotted seat formed along its entire length.

34. The bioartificial organ according to claim 33, wherein said seat has a central portion which is connected, along its entire length, to the axial cavity of said shaft-like element, the leading edge of said sheet-like distribution grid that forms said third layer being inserted in said central portion.

35. The bioartificial organ according to claim 26, wherein each capillary fiber is constituted by a tube-like segment of microporous material which is folded in a U-like shape substantially at the centerline so as to form two straight and parallel branches in s which the openings of the matching free ends lead into a corresponding collection chamber for plasma or ultrafiltrate.

36. The bioartificial organ according to claim 35, wherein said microporous material has pores with a diameter between 0.10 and 0.50 microns.

37. The bioartificial organ according to claim 35, wherein said microporous material is constituted by polyethersulfone.

38. The bioartificial organ according to claim 26, wherein the distribution density and the diameter of said capillary fibers are constant over the entire extension of said coil that constitutes said cell support and culture structure.
